(19) United States
(12) Patent Application Publication
(10) Pub. No.: US 2021/0308037 A1
(43) Pub. Date: Oct. 7, 2021

(54) Foot'e shea butter Moisturizer Glaze

(71) Applicant: Necho Gene Foot'e, Pittsburg, CA (US)

(72) Inventor: Necho Gene Foot'e, Pittsburg, CA (US)

(21) Appl. No.: 16/840,390

(22) Filed: Apr. 5, 2020

(51) Int. Cl. A61K 8/9789; A61Q 5/12; A61Q 19/00; A61K 8/98; A61K 8/9794; A61K 8/92

(52) U.S. Cl. CPC A61K 8/9789; A61Q 5/12; A61Q 19/007; A61K 8/987; A61K 2800/87; A61K 8/922; A61K 8/927; A61K 2236/10; A61K 2236/31; A61K 8/9794

(57) ABSTRACT

The present invention comprises a hair conditioner-scalp stimulator solution made of liquid extracts from Shea butter, beeswax, avocado oil, coconut oil, jojoba oil, pomegranate oil, sweet almond oil, argan oil, grape seed oil and red pimento oil.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

### Field of the Invention

The present invention relates to a solution to be applied topically to the human scalp in order to encourage hair growth, well-conditioned hair, control of dandruff, and overall health of the scalp. The present invention accomplishes these goals through a mixture of ingredients precisely proportioned to achieve a unique chemical composition that brings about maximum results. The ingredients in the solution include shea butter, beeswax, Avocado oil, coconut oil, jojoba oil, pomegranate oil, sweet almond oil, argan oil, grape seed oil and red pimento oil.

### Description of the Related Art

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Topical solutions for hair growth and overall scalp health are, and always will be, in high demand. Hair loss afflicts huge numbers of men and women, and inspires in its sufferers an ardent desire to solve the problem. This desire can be seen by the large number of inventions that have been created to stop hair loss. There have truly been a myriad of patented products to promote scalp health and generate the growth of hair, yet, as in any field of application; there is always room for improvement. This is where the present invention comes in.

Various natural ingredients have been used as hair conditioners and the like as evidenced by the following prior art. U.S. Pat. No. 41,791 to Smith used lemon juice, castor oil, alcohol, oil of rose, water, burdock root and balsam as a compound for the hair. U.S. Pat. No. 50,901 to Carr describes a hair restorative made with juice of onion, alcohol and oils of rosemary, bergamot and lemon.

U.S. Pat. No. 75,806 to Sterns discloses a hair dressing compound formed of coconut fat and castor oil mixed in alcohol. U.S. Pat. No. 116,562 to Calabro discloses a hair remedy formed of lemon juice, olive oil and powdered sulfur. U.S. Pat. No. 130,720 to Johnson describes a hair restorative made from the ashes of willow, ashes of leather, coconut butter, tallow and white grape wine.

U.S. Pat. No. 1,378,731 to Silvera describes a hair restorer using coconut oil. U.S. Pat. No. 3,809,747 to Tuscano discloses a hair and scalp treatment using chili pepper and lemon. U.S. Pat. No. 3,824,304 to Villanueva is directed to a hair conditioner using coconut oil, sweet orange, capsicum juice and tincture of iodine.

None of the prior art methods have been found to be completely suitable to meet these needs and are cumbersome. The present invention provides hair conditioner scalp stimulator to control scalp itch and dandruff, lessens hair loss, and provides thicker hair with more body and increased sheen. The solution also controls scalp oil and stimulates growth of dormant hair the overall combination of these features is nowhere disclosed in the prior art cited above which appears to be representative of the general art in this area although it is not intended to be an all-inclusive listing of pertinent prior art patents.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

In order to solve the above problems, the proposed invention comprises of a hair & scalp conditioner and Moisturizer that leaves hair healthy silky, shiny, sheen and has vitamins and nutrients.

Another object of the present invention relates to the composition which includes shea butter, Moisturizer conditioner glaze, essential oil blend is used on hair and scalp. Shea butter is melted down to oil under medium heat via stove or microwave then it is strained and filtered with a strainer through filter paper to make sure the shea butter is clean then the beeswax is melted down to oil and these oils are add by teaspoons or tablespoon or ounces depending on the batch size other ingredients include Avocado oil, coconut oil, jojoba oil, pomegranate oil, sweet almond oil, argan oil, grapeseed oil, red pimento oil, add fragrance essential oil then it's all whisked all together under medium heat and then poured into individual 2 oz tin cans and then placed in refrigerator for 1 hr to solidify for a constant texture and the set out in room temperature. After this process the product is ready.

The hair conditioner, scalp stimulator may be used at room temperature, or it may be refrigerated. The solution is applied directly to the scalp. Preferably, only small quantity is necessary and then are massaged into the scalp briskly for one or two minutes to ensure that the solution penetrates the scalp and hair. After the solution is applied it dries in a non-sticky manner and may be kept on the scalp overnight.

Some of the benefits of the present invention, besides the stimulation of hair growth, are moisturizing and conditioning the user's hair, controlling dandruff and itchy scalp, treating psoriasis, repairing damaged hair, controlling frizzy hair, giving hair a smooth look and feel, minimizing hair shedding, strengthening and thickening the hair, making hair softer and more manageable, building hair body, and restoring luster and shine to the hair. The solution is non-greasy and for all types of hair.

Another object of present invention is to provide a rinse conditioner which imparts superior conditioning effects, softness and manageability, static control, ease of combing and styling and long holding power.

Another object of the present invention is to provide an economical hair rinse condition containing a minimum concentration of essential active ingredients.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 a front views of the invention.

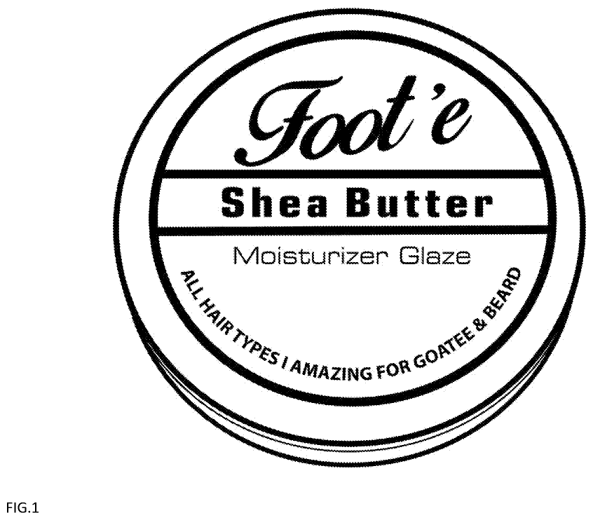

FIG. 2 is a box open view of the invention.

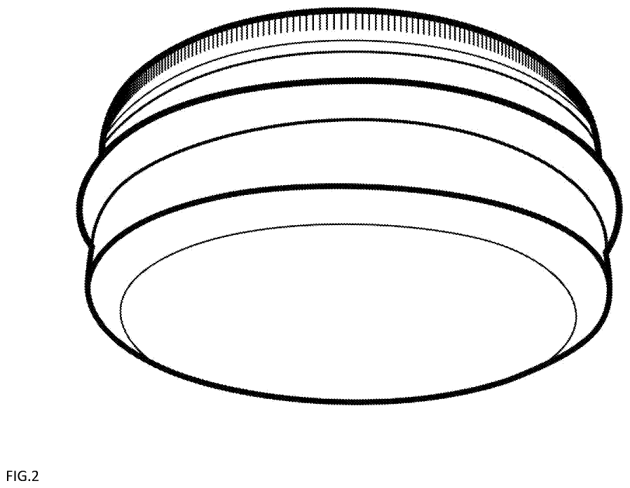

FIG. 3 is a side view of the invention

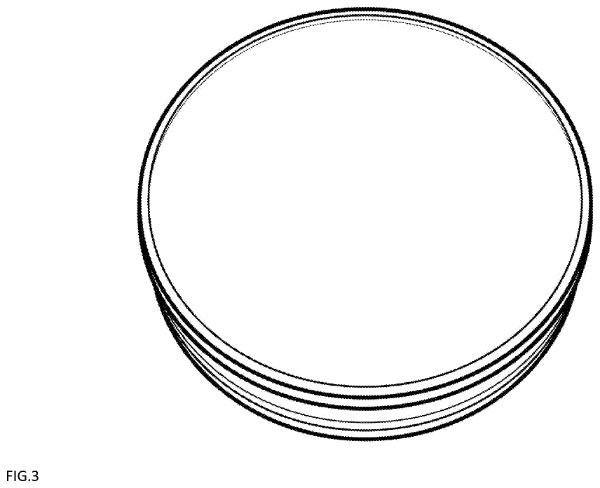

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

The present invention is a hair conditioner and scalp stimulant made of shea butter, beeswax, Avocado oil, coconut oil, jojoba oil, pomegranate oil, sweet almond oil, argan oil, grape seed oil and red pimento oil for application to the hair and scalp. The hair conditioner and scalp stimulant of the present invention is made from organic substances available from natural growing fruit trees. The solution mixture is generally clear with a slight skin color and exhibits a pleasant smell.

The hair conditioner in accordance with the invention may be in the form of a pourable lotion or a smooth cream. Further, the final product may have any suitable viscosity so long as it is appropriate for the final form selected, e.g., a pourable lotion, a thick or viscous lotion or a cream.

The present invention relates to the composition which includes shea butter, Moisturizer conditioner glaze, essential oil blend is used on hair and scalp. Shea butter is melted down to oil under medium heat via stove or microwave then it is strained and filtered with a strainer through filter paper to make sure the shea butter is clean then the beeswax is melted down to oil and these oils are add by teaspoons or tablespoon or ounces depending on the batch size other ingredients include Avocado oil, coconut oil, jojoba oil, pomegranate oil, sweet almond oil, argan oil, grapeseed oil, red pimento oil, add fragrance essential oil then it's all whisked all together under medium heat and then poured into individual 2 oz tin cans and then placed in refrigerator for 1 hr to solidify for a constant texture and the set out in room temperature. After this process the product is ready.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1) A hair & scalp conditioner & Moisturizer comprising portions of liquid extracts derived from shea butter, beeswax, avocado oil, coconut oil, jojoba oil, pomegranate oil, sweet almond oil, argan oil, grape seed oil and red pimento oil.

2) The ingredients shea butter is melted down to oil under medium heat via stove or microwave then it is strained and filtered with a strainer through filter paper to make sure the shea butter is clean, then the beeswax is melted down to oil and these oils are added by teaspoons or tablespoon or ounces depending on the batch size, all other ingredients are whisked together under medium heat and then poured into individual tin cans and then placed in the refrigerator for 1 hr to solidify for a constant texture and the set out in room temperature to pack the product.

3) The invention promotes healthy hair lifestyle hair maintenance damage prevention and repair restoration.
